(19) United States
(12) Patent Application Publication
(10) Pub. No.: US 2025/0021913 A1
(43) Pub. Date: Jan. 16, 2025

(54) SYSTEMS AND METHODS FOR WEIGHTING, SCORING, AND RANKING ENTITIES WITH RESPECT TO SPECIALTIES

(71) Applicant: U.S. News & World Report, L.P., New York, NY (US)

(72) Inventor: Bentsion R. HARDER, Bethesda, MD (US)

(21) Appl. No.: 18/771,959

(22) Filed: Jul. 12, 2024

Related U.S. Application Data
(60) Provisional application No. 63/513,314, filed on Jul. 12, 2023.

(51) Int. Cl. G06Q 10/0639; G06Q 10/067; G16H 40/20

(52) U.S. Cl. CPC G06Q 10/06393; G06Q 10/067; G16H 40/20

(57) ABSTRACT

Systems and methods are disclosed for ranking entities based on performance evaluation with respect to specialties. The method includes collecting a plurality of data associated with entities from source(s); selecting, based on the plurality of data, entities for which performance evaluation is conducted with respect to specialties; for each of the specialties: determining, using one or more models, an overall score for each of the selected entities based on performance score(s) determined for performance component(s), wherein the performance component(s) include: a structural component, a process/expert opinion component, an outcome component, a patient experience component, or a public transparency component, wherein each component includes performance indicator(s); generating a rank for each of the selected entities based on the overall score determined for each of the selected entities; and causing a display of the rank for the selected entities in association with the specialties in a user interface of a device.

### CROSS-REFERENCE TO RELATED APPLICATION

This application is a non-provisional of, and claims the benefit of priority to U.S. Provisional Application No. 63/513,314, filed on Jul. 12, 2023, the disclosure of which is incorporated herein in its entirety by reference.

### TECHNICAL FIELD

This present disclosure relates generally to the field of data processing and advanced analytics. In particular, the present disclosure relates to a data-driven method for scoring and ranking a plurality of entities.

### BACKGROUND

Ranking entities (e.g., hospitals) may play a crucial role in providing transparency and aiding informed decision-making for patients, healthcare providers, and policymakers alike. However, current methodologies often face significant technical challenges as they oversimplify complex healthcare metrics or rely on limited datasets that fail to capture the full spectrum of the performance of one or more entities. For example, existing approaches may depend on simplistic metrics which may not adequately capture the complexities of healthcare quality. These metrics often suffer from biases, inadequate risk adjustment, and incomplete datasets, leading to skewed rankings that fail to provide a comprehensive picture of an entity's performance. Moreover, traditional methods typically lack robust methods for integrating and analyzing diverse data sources, such as real-time clinical outcomes, operational efficiency metrics, and patient-reported outcomes. This fragmented approach hinders the ability to accurately assess the entity's quality across multiple dimensions and can obscure meaningful differences in care delivery. There is a pressing need for a new approach that leverages advanced statistical models, machine-learning algorithms, and big data analytics to handle the multidimensional nature of healthcare quality assessment.

### SUMMARY OF THE DISCLOSURE

The present disclosure solves the technical challenges typically encountered during the use of a conventional method, such as those discussed above. Specifically, the present disclosure solved the technical challenges by training a machine-learning model to score and rank one or more entities.

In some embodiments, a computer-implemented method includes: collecting, using one or more processors, a plurality of data associated with one or more entities from one or more sources; selecting, using the one or more processors and based on the plurality of data, one or more entities for which performance evaluation is conducted with respect to one or more specialties; for each of the one or more specialties: determining, using the one or more processors and using one or more models, an overall score for each of the one or more selected entities based on one or more performance scores determined for one or more performance components, wherein the one or more performance components include one or more of: a structural component, a process/expert opinion component, an outcome component, a patient experience component, or a public transparency component, wherein each component includes one or more performance indicators; generating, using the one or more processors, a rank for each of the one or more selected entities based on the overall score determined for each of the one or more selected entities; and causing, using the one or more processors, a display of the rank for the one or more selected entities in association with the one or more specialties in a user interface of a device.

In some embodiments, a system for one or more processors of a computing system; and at least one non-transitory computer readable medium storing instructions which, when executed by the one or more processors, cause the one or more processors to perform operations including: collecting a plurality of data associated with one or more entities from one or more sources; selecting, based on the plurality of data, one or more entities for which performance evaluation is conducted with respect to one or more specialties; for each of the one or more specialties: determining, using one or more models, an overall score for each of the one or more selected entities based on one or more performance scores determined for one or more performance components, wherein the one or more performance components include one or more of: a structural component, a process/expert opinion component, an outcome component, a patient experience component, or a public transparency component, wherein each component includes one or more performance indicators; generating a rank for each of the one or more selected entities based on the overall score determined for each of the one or more selected entities; and causing a display of the rank for the one or more selected entities in association with the one or more specialties in a user interface of a device.

In some embodiments, a non-transitory computer readable medium storing instructions which, when executed by one or more processors of a computing system, cause the one or more processors to perform operations including: collecting a plurality of data associated with one or more entities from one or more sources; selecting, based on the plurality of data, one or more entities for which performance evaluation is conducted with respect to one or more specialties; for each of the one or more specialties: determining, using one or more models, an overall score for each of the one or more selected entities based on one or more performance scores determined for one or more performance components, wherein the one or more performance components include one or more of: a structural component, a process/expert opinion component, an outcome component, a patient experience component, or a public transparency component, wherein each component includes one or more performance indicators; generating a rank for each of the one or more selected entities based on the overall score determined for each of the one or more selected entities; and causing a display of the rank for the one or more selected entities in association with the one or more specialties in a user interface of a device.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of a system for dynamically weighting, scoring, and ranking one or more entities, according to aspects of the disclosure.

FIG. 2 is a flowchart of a process for ranking entities based on performance evaluation with respect to specialties, according to aspects of the disclosure.

FIG. 3 is a flowchart of a process for determining eligibility and ranking of community hospitals in a data-driven ranking system, according to aspects of the disclosure.

FIG. 4 shows an example machine-learning training flow chart.

FIG. 5 illustrates an implementation of a computer system that executes techniques presented herein.

### DETAILED DESCRIPTION OF EMBODIMENTS

This present disclosure relates generally to the field of data processing and advanced analytics. In particular, the present disclosure relates to a data-driven method for scoring and ranking a plurality of entities.

Ranking entities (e.g., hospitals) may present significant technical challenges due to the inherent complexity of healthcare data and the multifaceted nature of an entity's performance. One issue is the integration and standardization of diverse data sources, including clinical outcomes, patient demographics, operational metrics, and patient-reported experiences. These datasets often vary in format, granularity, and reliability, making it difficult to create a unified and comprehensive ranking system. Moreover, existing methodologies frequently rely on simplistic or unweighted aggregation of these metrics, which can obscure important nuances and lead to inaccurate assessments of the entity's performance. The variability in data quality and completeness further exacerbates these issues, requiring sophisticated data preprocessing and normalization techniques to ensure fairness and comparability.

Conventional methodologies are often technically deficient due to their reliance on limited and static datasets. Many traditional systems prioritize easily quantifiable metrics without adequately accounting for context or underlying factors that may influence those outcomes. For example, hospitals serving high-risk populations or those with complex medical needs may be unfairly penalized despite providing high-quality care. Current methods also frequently lack robust risk adjustment mechanisms to account for patient severity and comorbidities, leading to skewed rankings that do not reflect true performance differences. Additionally, the absence of real-time data integration limits the ability of these systems to provide up-to-date and relevant insights, further diminishing their utility for stakeholders seeking accurate and actionable information.

Current methodologies are limited in their technical capacity to handle and process the vast volumes of unstructured data generated in healthcare settings. Traditional systems often rely primarily on structured data, such as numerical and categorical data from electronic health records (EHRs), and may ignore valuable unstructured data sources like clinical notes, imaging reports, and patient feedback. These unstructured data contain rich and nuanced information that can provide deeper insight into hospital performance. The inability to integrate and analyze unstructured data may lead to incomplete and potentially biased rankings. This technical deficiency underscores the need for more sophisticated data processing capabilities that can leverage the full spectrum of available healthcare data, ensuring a more accurate and holistic assessment of hospital quality.

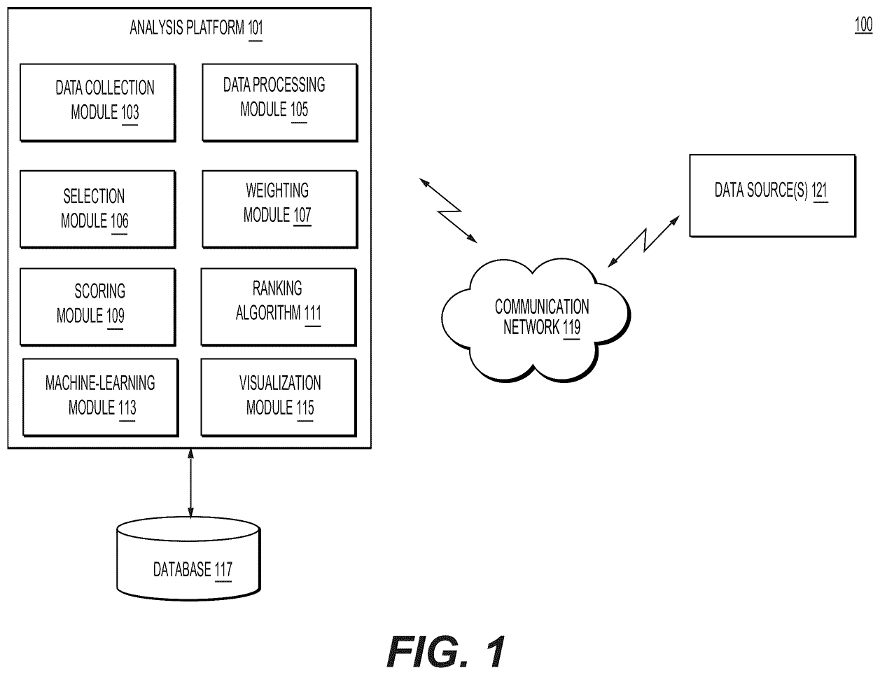

System 100 of FIG. 1 may address the limitations of the conventional methods through a multifaceted approach that leverages advanced data processing, machine-learning, and sophisticated statistical techniques. Unlike the traditional methodologies that often rely on static models and limited data points, the system 100 may dynamically incorporate a vast array of data sources, including publicly available indicators, specialized datasets (e.g., Medicare Beneficiary Summary File (MBSF), Limited Data Sets Standard Analytical Files (LDS SAF), and American Hospital Association (AHA) surveys), and detailed clinical outcomes. By utilizing comprehensive risk adjustment variables (e.g., age, sex, comorbidities, and socioeconomic status) the system 100 may ensure a more accurate comparison of hospitals, effectively normalizing for patient demographics and health conditions. In one example, system 100 may rank hospitals by assigning points based on their performance across various specialties and procedures, deducting points for below-average ratings, and ultimately ranking hospitals regionally and metro-wise. This process may reduce biases inherent in the conventional methods and may provide a more equitable assessment of hospital performances.

Furthermore, the system 100 may employ advanced ranking algorithms and machine-learning models to determine the optimal weighting of quality indicators, addressing the issues in constructing composite ratings. These algorithms may empirically derive the significance of each indicator based on its predictive value and may adjust for measurement errors due to incomplete risk adjustments or random variation from low sample sizes. System 100 may implement rigorous inclusion and exclusion criteria that may ensure that only hospitals meeting high standards in specific specialties and procedures are considered for ranking. By continuously updating and refining the models with new data, the system 100 may maintain relevance and accuracy, unlike status models that quickly become outdated. The inclusion of process and structural measures, alongside outcome-based metrics, may offer a holistic view of hospital quality, encompassing factors like nurse staffing ratios, compliance with treatment protocols, and accreditation status. This comprehensive and adaptive approach may ensure that the rankings are not only more precise but also more reflective of actual hospital performance.

FIG. 1 is a diagram showing an example of a system for dynamically weighting, scoring, and ranking one or more entities, according to aspects of the disclosure. FIG. 1 includes the system 100 that comprises analysis platform 101, a database 117, a communication network 119, and data source(s) 121.

In one embodiment, the analysis platform 101 is a platform with multiple interconnected components. The analysis platform 101 includes one or more servers, intelligent networking devices, computing devices, components, and corresponding software for dynamically weighting, scoring, and ranking one or more entities based on pre-defined criteria.

The analysis platform 101 may gather, in real-time or near real-time, extensive information on hospitals, including their characteristics, services, and performance metrics from multiple sources (e.g., national databases, hospital records, surveys, and other relevant repositories). Once the data is collected, the analysis platform 101 may process the collected data to determine the eligibility of hospitals. This may involve applying predefined criteria to assess whether each hospital meets the necessary conditions to be included in the ranking process. The criteria may include factors such as membership in professional organizations (e.g., Council of Teaching Hospitals), affiliations with accredited medical schools, hospital bed count, and availability of advanced technologies. This eligibility determination may ensure that only hospitals meeting specific standards are considered for ranking.

The analysis platform 101 may assign one or more weights to various performance criteria (e.g., entity structures, entity processes, staffing levels, advanced technology availability, patient volume, or patient services). The assignment of weights may be based on the relative importance of each criterion in evaluating hospital performance. By weighting the criteria, the method ensures that more critical factors have a greater influence on the final ranking. The analysis platform 101 may score each hospital based on its performance in the weighted criteria. The scoring process may quantify the hospital's performance across multiple dimensions, such as healthcare delivery outcomes and patient satisfaction. This step converts qualitative and quantitative performance metrics into a standardized scoring system, allowing for objective comparison between hospitals. In one instance, the analysis platform 101 may calculate scores for each hospital based on scoring criteria (e.g., survival rates, discharge from home rates, patient experience, readmission rate, staff satisfaction, or cost efficiency). The analysis platform 101 may evaluate the performance of the hospitals in the weighted criteria to generate scores on survival rates, discharge from home rates, patient experience, readmission rate, staff satisfaction, or cost efficiency.

The analysis platform 101 may utilize the calculated scores to generate a ranking of the hospitals. This ranking is based on the overall scores derived from the weighed performance criteria. Hospitals may be positioned relative to each other within a defined healthcare performance spectrum. The ranking may provide a clear and ordered list of hospitals, highlighting their comparative performance across the evaluated dimensions. The analysis platform 101 may generate a presentation of the ranked data in the user interface of a device. This presentation is designed to be accessible and informative, facilitating analysis and decision-making by healthcare providers, policymakers, and patients. The ranked data may be displayed in various formats, such as lists, charts, or dashboards, providing users with a comprehensive view of hospital performance and enabling them to make informed choices based on ranking.

In one instance, the analysis platform 101 may include a data collection module 103, a data processing module 105, a selection module 106, a weighting module 107, a scoring module 109, a ranking algorithm 111, a machine-learning module 113, and a visualization module 115, or any combination thereof. As used herein, terms such as “component” or “module” generally encompass hardware and/or software, e.g., that a processor or the like used to implement associated functionality. It is contemplated that the functions of these components are combined in one or more components or performed by other components of equivalent functionality.

In one instance, the data collection module 103 may collect relevant data associated with the hospitals through various data collection techniques. For example, the data collection module 103 may use a web-crawling component to access various databases (e.g., data source(s) 121) to collect the relevant data. In one instance, the data collection module 103 may include various software applications (e.g., data mining applications in Extended Meta Language (XML)) that automatically search for and return relevant data associated with the hospitals. Through seamless interaction with various databases, the data collection module 103 may capture real-time data updates, ensuring data accuracy and completeness, minimizing errors and enhancing the reliability of the collected data. In one example, the data collection module 103 may collect comprehensive data including hospital records, health databases, patient surveys, American Nurse Association (ANA) surveys, and accreditation reports from the data source(s) 121. The hospital records may provide insights into infrastructure, patient demographics, and medical procedures, while the health databases may offer statistical data on patient outcomes and treatment efficacy. The patient and ANA surveys may contribute valuable feedback on care quality and staff satisfaction, respectively. The accreditation reports may detail compliance with rigorous industry standards, ensuring hospitals meet essential criteria for inclusion in the rankings. By synthesizing this varied data landscape, the data collection module 103 may ensure a robust foundation for subsequent analysis and scoring. In one example, plurality of data objects received from one or more of the patients, entity leaders, or other stakeholders include results from various surveys, for example:
Surveys: Surveys such as patient satisfaction surveys and organizational assessments like the American Nurse Association (ANA) surveys may capture feedback from patients, healthcare professionals, and stakeholders. These surveys may evaluate aspects such as patient experience, staff satisfaction, and overall organizational performance. In the weighting process, survey results may be assigned significant weight to reflect their role in assessing subjective aspects of hospital case quality and organizational effectiveness. Hospitals that receive positive feedback through surveys may receive higher scores, reflecting their commitment to patient-centered care and organizational excellence.

In one instance, the data processing module 105 may process the collected raw data, ensuring consistency and comparability across different hospitals and datasets. The data processing module 105 may perform data cleaning on the raw data, by identifying and rectifying anomalies, such as missing values, duplicates, and outliers using sophisticated algorithms. The data processing module 105 may implement transformation processes to convert the data into a standardized format, employing techniques like data parsing and encoding to ensure interoperability between different data sources. The data processing module 105 may normalize the data to ensure that disparate data metrics are scaled to a common range, thereby allowing fair comparisons. Advanced techniques like natural language processing (NLP) may be employed to extract meaningful information from unstructured data sources, such as physician's notes and patient feedback.

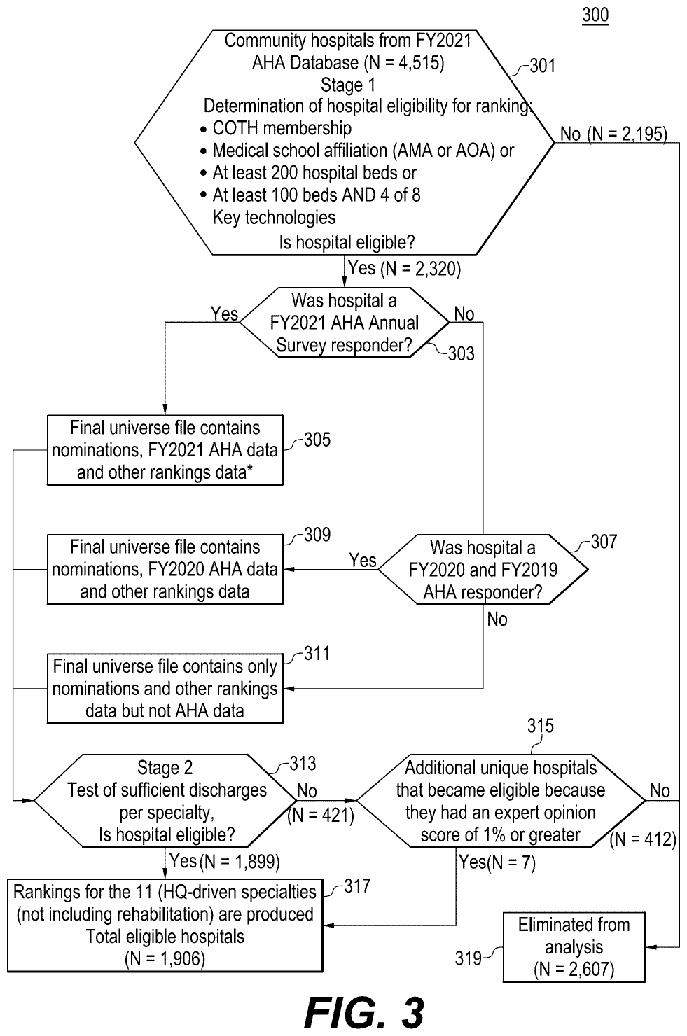

In one instance, the data processing module 105 may determine the eligibility of hospitals for inclusion in the ranking system. The data processing module 105 may systematically analyze the data collected from various sources to evaluate whether the hospitals meet specific criteria. These criteria may include membership in the Council of Teaching Hospitals (COTH), affiliation with an accredited medical school (either AMA or AOA), having at least 200 beds, or possessing at least 100 beds and four out of eight key advanced technologies. Hospitals that meet these criteria are deemed eligible and move forward in the ranking process. This process is represented in FIG. 3.

In one instance, all structural measure values may be normalized prior to weighting. Normalization may transform index values into a distribution between 0 and 1 based on the range of possible values for a given measure. Normalizations may be done separately for each specialty. Equation (1) is the formula for normalization:

$$Normalized Value = ( Xi - Minimumi ) / ( Maximumi - Minimumi )$$

Xi=the value for measure i,
Maximumi=the highest possible value for measure i and
Minimumi=the lowest possible value for measure i.

For example, the Advanced Technologies index for Cancer is worth a maximum of 8 points. If a given hospital received 5 out of 8 points, the normalized value for the Advanced Technologies index in Cancer would be (5-0)/(8-0)=0.63. For all structural measures, other than Number of Patients and Nurse Staffing, the lowest possible value is 0 even when the lowest observed value is greater than 0. For Number of Patients and Nurse Staffing, the lowest possible value was made equal to the lowest observed value and the highest possible value was made equal to the highest observed value.

In one embodiment, the selection module 106 may select entities (e.g., hospitals) for the purpose of ranking based on one or more of:
1. Structural Characteristics: Hospital structure may refer to the physical infrastructure, facilities, and resources available within the hospital. This criterion may assess factors such as the number of beds, specialized units (e.g., ICU, surgical suites), availability of advanced medical equipment, and overall facility maintenance. In the weighting process, hospital structure may be assigned significant weight to reflect its foundational role in supporting patient care and treatment outcomes. Hospitals with modern facilities and adequate infrastructures may likely receive higher scores, indicating their capacity to deliver comprehensive and effective healthcare services.
2. Volume: The weighting module 107 may measure the number of patients treated by the hospital within a specific period, including both inpatient admissions and outpatient visits. This criterion may provide insight into the hospital's capacity, workload, and patient demand. Weighting for patient volumes may consider the hospital's ability to manage a large volume of patients efficiently while maintaining quality of care and patient satisfaction. Hospitals with a high patient volume may receive higher scores, indicating their ability to handle diverse healthcare needs and maintain operational excellence.
3. Discharge characteristics: The discharge characteristics may indicate data relating to patient discharges, including discharge rates, average length of stay, discharge destination (e.g., home, rehabilitation centers, or other medical facilities), and readmission rates. The discharge to home measure assesses how well a hospital does at managing to discharge patients to home rather than sending them on to another acute or post-acute care setting following hospitalization. In one instance, if the discharge characteristic for an entity is below a pre-determined threshold, the entity may be selected if nominated by a certain percentage or number of providers and/or provider systems.

In one instance, a performance evaluation may be conducted by the scoring module 109 for the selected entities with respect to one or more specialties.

In one instance, the weighting module 107 may assign relative importance to various criteria based on their impact on hospital performance and patient outcomes. The weighting module 107 may apply advanced algorithms to calculate weighted scores for each criterion, integrating factors such as hospital structures, patient outcomes, nurse staffing levels, advanced technology availability, patient volume, and outcomes. By applying these weights, the weighting module 107 may reflect the relative significance of each criterion in contributing to overall hospital quality and performance.

In one instance, the scoring module may evaluate various performance criteria, such as survival rates, discharge to home rates, patient experience scores, readmission rates, and other clinical outcomes. These outcomes may reflect the effectiveness of hospital care and treatment protocols in achieving positive results for patients. When comparing outcomes such as mortality between hospitals, adjusting for differences in the patients treated at each hospital is critical. These adjustments need to take into account not only the principal condition for which the patient is being treated but also other comorbidities and characteristics that may affect outcomes. For instance, a hospital with a 35% death rate might be superior to a hospital with a 10% death rate, if most of the patients at the first hospital are of high risk (i.e., expected to die) and most of the patients at the second hospital are of fairly low risk. In one instance, the scoring module may utilize multilevel logistic regression models to adjust for differences in case mix between hospitals.

In one example, changes over the years have addressed specific issues in calculating mortality. These changes have addressed either specialty-specific issues (such as defining a specific population to use in Geriatrics as opposed to using all cases) or more general issues that can affect mortality outcomes (such as excluding transfers). Brief descriptions of these special considerations are:
1. Redefining the Geriatrics patient population: This involves updating the criteria and characteristics used to identify and classify elderly patients and recognizing the diverse and complex nature of this demographic. By adopting a more comprehensive and patient-centered definition, healthcare providers can enhance the quality of care, improve health outcomes, and better address the unique needs of the geriatric population.
2. Excluding transfers from mortality calculations: Adjusting how deaths are calculated more accurately reflects the impact of care provided within a hospital. Typically, when a patient is transferred to another facility and subsequently passes away, the death is attributed to the initial hospital in mortality statistics. However, for more precise assessment of hospital's performance, excluding these cases from mortality calculations may provide a clearer picture of a hospitals direct impact on patient outcomes.
3. Standardizing on 30-day mortality: Focusing on death that occurs within a specific timeframe after hospital admission, typically within 30 days. This metric is used to standardize mortality rates across hospitals and account for variations in patient populations, severity of illness, and treatment protocols.
4. Adjustment for socioeconomic status and risk: Socioeconomic status encompasses factors like income, education level, and access to resources, which may significantly impact health outcomes independent of medical care quality. Risk adjustment methodologies aim to statistically normalize or standardize outcomes data to reflect the differences in patient populations' socioeconomic backgrounds and health risks. By adjusting for socioeconomic status and risk factors, hospitals may ensure fair comparisons and more accurately assess their performance relative to peers.
5. Update to the calculation of Survival and Discharge to Home: A new risk-adjustment approach for the Survival and Discharge to Home outcomes that move away from the observed to expected ratios (OER) to ‘random effect’ (RE) models, which can be thought of as a hospital level off-set. They represent the risk difference between a hospital and all hospitals in a given specialty, discounted by the reliability of that difference. The reliability is based on the volume of cases in a hospital, which means that if a hospital has 500 cases and 0 deaths, they would have a better RE, and thus better mortality score, than a hospital with 50 cases and 0 deaths; previously, these hospitals would have had the same OER of 0. The rationale for this is that in hospitals where there are more observations, there is higher certainty that the observed results are real and not due to statistical noise.

In one embodiment, the scoring module may determine an overall score for the selected entities based on one or more performance scores determined for one or more performance components. The performance components may include one or more of:
1. Structural component: This may encompass the physical and organizational attributes that may define a healthcare facility's capacity and capability to deliver quality care. This may include factors such as the hospital's infrastructure, facilities, and resources, such as the number of beds, availability of advanced medical technologies and specialized units (e.g., ICU, trauma centers).
2. Processes/Expert Opinion Component: Processes in healthcare may refer to the procedures, protocols, and workflows that govern how medical care is delivered within a hospital. This criterion may evaluate the efficiency, effectiveness, and adherence to clinical guidelines in various aspects of patient care, administrative tasks, and operational management. Key elements may include average wait times, adherence to clinical protocols, or implementation of quality improvement initiatives. In the weighting process, the hospital's processes may be assigned a significant weight to reflect their impact on patient safety, treatment outcomes, and overall hospital performance. Hospitals with streamlined processes, effective quality control measures, and a commitment to continuous improvement may receive higher scores, indicating their ability to deliver consistent and high-quality healthcare services.

Expert opinion within a data-driven hospital ranking system may encompass insights and evaluations provided by medical professionals, healthcare specialists, and industry experts who possess deep knowledge and experience in hospital operations and patient care. This criterion may leverage surveys, peer reviews, and expert assessment to gauge hospital performance beyond quantitative metrics. Expert opinions may consider factors such as clinical expertise, leadership quality, research contributions, and the hospital's reputation within the healthcare community. In the weighting process, expert opinion may be assigned significant weight to reflect its role in assessing intangible yet critical aspects of hospital excellence, leadership, and innovation. Hospitals that receive positive expert opinions and high ratings from healthcare professionals may receive higher scores, highlighting their recognition and respect within the medical community.
3. Output component: This may encompass a wide range of metrics that reflect the effectiveness, efficiency, and impact of medical treatments, interventions, and care protocols. Key indicators within the output component may include patient outcomes such as mortality, complication rates, readmission rates, infection rates, and functional status improvements. These metrics may provide critical insights into how well hospitals are managing and improving patient health outcomes, thereby indicating the quality of clinical care and the success of treatment strategies.
4. Patient experience component: This may include various aspects of patient interactions, satisfaction, and perceptions throughout their healthcare journey. Key indicators within this component may include communication with healthcare providers, responsiveness of hospital staff, pain management, cleanliness and comfort of facilities, and involvement in care decisions. Evaluating the patient experience component may provide insights into how well hospitals are meeting the emotional, informational, and support needs of patients, which are crucial for patient-centered care.
5. Public transparency component: Transparency via clinical registries and other public transparency programs can facilitate informed decision making by patients, which in turn may boost patient engagement in their healthcare. Transparency also creates opportunities for researchers to externally validate or critically evaluate the results of hospital rankings. Moreover, it demonstrates a public commitment on the part of the participating hospitals to the process of pursuing quality improvement.

In one embodiment, the outcome component may include one or more of the following performance indicators:
1. Mortality rate: The mortality rate may reflect the proportion of patients who die during a specified period of time. This metric may evaluate the effectiveness of clinical treatments and interventions, as well as the overall quality of care provided by the hospital. A high mortality rate may indicate potential issues with patient safety, quality of care, or the severity of cases treated. Conversely, a low mortality rate may suggest that the hospital is successfully managing patient conditions and minimizing fatal outcomes.
2. Discharge rate: Discharge rate may measure the frequency at which patients are released from the hospital after receiving care, typically indicating successful treatment and recovery. A high discharge rate may suggest effective and timely patient care, allowing hospitals to accommodate new patients and manage resources efficiently.
3. Measure of outpatient complication: The measure of patient outcome may track the incidence of adverse events or medical issues that arise during or after outpatient treatments or procedures. A high rate of outpatient complications may indicate potential deficiencies in clinical practices, patient management, or procedural protocols.

In one embodiment, the structural component may include one or more of the following performance indicators:
1. Advanced technology availability: The weighting module 107 may assess the hospital's access to and utilization of cutting-edge medical equipment, technologies, and treatment modalities. This criterion may include innovations such as robotic surgery systems, transplant services, advanced imaging technologies (e.g., MRI, CT scans), prosthetic and orthotic services, telemedicine capabilities, computer-assisted orthopedic surgery, electrodiagnostic services, diagnostic radioisotope services, electrodiagnostic services, endoscopic retrograde cholangiopancreatography, intensity-modulated radiation therapy (IMRT), endoscopic ultrasound, full-field digital mammography, multislice spiral computed tomography, shaped-beam radiation, single-photon-emission CT, simulated rehabilitation environment, or stereotactic radiosurgery. In the weighting process, advanced technology availability is often assigned a significant weight to recognize its impact on diagnostic accuracy, treatment effectiveness, and patient outcomes. Hospitals that invest in and integrate advanced technologies into patient care may receive higher scores, demonstrating their commitment to delivering optimal healthcare services.
2. Number of patients: This metric may reflect the volume and diversity of medical cases managed by the hospital, providing insights into its operational efficiency and ability to handle varying levels of patient demand. Hospitals with higher patient volumes often demonstrate robust clinical experience and expertise across different specialties, as well as a broader scope of services and resources to meet community healthcare needs.
3. Outpatient volume: This metric may assess a hospital's ambulatory services and its capacity to manage non-emergency medical conditions efficiently. Higher outpatient volumes may indicate a robust outpatient department capable of handling a diverse range of medical needs, from routing consultations to specialized outpatient procedures. Hospitals with significant outpatient volume often emphasize accessibility, convenience, and continuity of care of patients.
4. Volume of care: This metric may reflect both inpatient and outpatient services, reflecting the breadth of healthcare interventions, treatments, and procedures performed within the facility. Hospitals with a high volume of care often demonstrate extensive clinical experience and expertise across various specialties, indicating their capability to handle complex medical cases and manage a diverse patient population effectively.
5. Nurse staffing levels: Nurse staffing levels may evaluate the ratio of nurses to patients, which may directly influence patient safety, quality of care, and overall hospital performance. Nurse staffing levels are a critical structural measure, as adequate nurse staffing is linked to better patient outcomes, including lower rates of complications, infections, and mortality. The weighting module 107 may evaluate the number of full-time equivalent (FTE) registered nurses available to care for the patients. The weighting module 107 may consider factors such as nurse workload, skill mix (e.g., registered nurses vs. nurse aides), and adherence to staffing guidelines. Hospitals with optimal nurse staffing levels, where nurses can provide attentive and skilled care, may receive higher scores, reflecting their commitment to patient welfare and safety.
6. Nurse staffing index: This is a specific metric that may calculate the ratio of FTE registered nurses to adjusted patient days. This ratio may provide a standardized measure of nurse availability relative to patient volume, offering insight into the hospital's capacity to provide adequate nursing care. Higher ratios may indicate better nurse staffing levels, which are associated with improved patient outcomes.
7. Trauma center: The trauma center designation may evaluate the hospital's capability to provide specialized care for trauma patients, ranging from minor injuries to critical emergencies. This criterion may consider factors such as the level of trauma care designation (e.g., Level I, Level II), availability of specialized trauma teams, and trauma care outcomes. In the weighting process, trauma center designation may be assigned a significant weight to recognize its critical role in managing life-threatening injuries and emergencies. Hospitals with higher trauma center designations and superior trauma care outcomes may receive higher scores, indicating their readiness to handle complex medical emergencies.
8. Volume measure for rehabilitation: A performance criterion used in the hospital ranking method to evaluate the extent and effectiveness of rehabilitation services provided by a hospital. This measure typically includes the number of patients treated in various rehabilitation programs, the range of rehabilitation services offered, and the overall capacity of the hospital's rehabilitation department. Higher volumes may indicate greater experience and expertise in delivering rehabilitation care, which may correlate with better patient outcomes. By assessing the volume measure for rehabilitation, the ranking methods can determine the hospital's proficiency in handling diverse and complex rehabilitation cases.
9. Patient services: To ensure a comprehensive and equitable ranking system, the weighting module 107 may assign weight to each specialized patient service based on its significance, demand, and impact on overall hospital quality and patient outcomes. In one example, patient services may include:
   (i) Alzheimer's center: A facility that cares for individuals with Alzheimer's disease and the patients' families through an integrated program of clinical services, research, and education.
   (ii) Arthritis treatment center: A center specifically equipped and staffed for diagnosing and treating arthritis and other joint disorders.
   (iii) Cardiac rehabilitation: A medically supervised program to help heart patients recover quickly and improve their overall physical and mental functioning in order to reduce the risk of another cardiac event or to keep a current heart condition from worsening.
   (iv) Cardiac intensive care unit: The unit is staffed with specially trained physicians and nursing personnel with specialty monitoring and support/treatment equipment for patients who, because of heart seizure, open-heart surgery, or other life-threatening conditions, require intensified, comprehensive observation and care.
   (v) Case management: A system of assessment, treatment planning, referral, and follow-up that may ensure the provision of comprehensive and continuous services and the coordination of payment and reimbursement for care.
   (vi) Employment support services: Services designed to support individuals with significant disabilities to seek and maintain employment.
   (vii) Enabling services: A program that is designed to help the patient access health care services by offering transportation services and/or referrals to local social services agencies.
   (viii) Fertility clinic: A specialized program set in an infertility center that provides counseling and education, as well as advanced reproductive techniques.
   (ix) Genetic testing/counseling: A service equipped with adequate laboratory facilities and directed by a qualified physician to advise parents and prospective parents on potential problems in cases of genetic defects.
   (x) Health research: Organized hospital research program in basic research, clinical research, community health research, and/or research on innovative health care delivery.
   (xi) Hemodialysis: Provision of equipment and personnel for the treatment of renal insufficiency on an inpatient or outpatient basis.
   (xii) Hospice: A program that provides care (including pain relief) and supportive services for the terminally ill and their families.
   (xiii) Infection isolation room: A single-occupancy room designed to minimize the possibility of infectious transmission, typically through the use of controlled ventilation, air pressure, and filtration.
   (xiv) Neurological services: Services provided by the hospital dealing with the operative and non-operative management of disorders of the central, peripheral, and autonomic nervous systems.
   (xv) Occupational health services: Services designed to protect the safety of employees from hazards in the work environment.
   (xvi) Pain-management program: A program that provides specialized care, medications, or therapies for the management of acute or chronic pain.
   (xvii) Palliative care: A program that provides specially trained physicians and other clinicians to relieve acute or chronic pain or to control symptoms of illness.
   (xviii) Patient-controlled analgesia: A system that allows the patient to control intravenously administered pain medicine.
   (xix) Patient education center: Written goals and objectives for the patient and/or family related to therapeutic regimens, medical procedures, and self-care.
   (xx) Patient representative services: Organized hospital services providing personnel through whom patients and staff can seek solutions to institutional problems affecting the delivery of high-quality care and services.
   (xxi) Physical rehabilitation outpatient services: Program providing medical, health-related, therapy, social, and/or vocational services to help people with disabilities attain or retain their maximum functional capacity.
   (xxii) Psychiatric services—psychiatric consultation-liaison services: Provides organized psychiatric consultation/liaison services to non-psychiatric hospital staff and/or departments on psychological aspects of medical care that may be generic or specific to individual patients.
   (xxiii) Psychiatry-geriatric service: A psychiatric service that specializes in the diagnosis and treatment of geriatric medical patients.
   (xxiv) Social work services: Organized services that are properly directed and sufficiently staffed by qualified individuals who provide assistance and counseling to patients and their families in dealing with social, emotional, and environmental problems associated with illness or disability, often in the context of financial or discharge planning coordination.
   (xxv) Support groups: A hospital-sponsored program that allows a group of individuals with common experiences or issues who meet periodically to share experiences, problems, and solutions in order to support each other.
   (xxvi) Translators: A service provided by the hospital to assist patients who do not speak English.
   (xxvii) Wound-management services: Services for patients with chronic and non-healing wounds that often result from diabetes, poor circulation, sitting or reclining improperly, and immunocompromising conditions.
10. ICU specialist: These professionals oversee the care of critically ill patients in intensive care units (ICUs), ensuring they receive comprehensive and specialized treatment. In the context of hospital ranking, the presence of an ICU specialist is a critical performance criterion, reflecting the hospital's capability to deliver high-quality critical care services and potentially influencing its overall ranking.
12. Accreditation from External Organization: Accreditation from external organization plays a critical role in the ranking method by providing an additional layer of validation for hospital performance. External organization may include:
   (i) National Cancer Institute (NCI) designated Cancer Center and/or American College of Surgeons (ACS) Commission on Cancer: Hospitals that are designated as NCI cancer centers or accredited by the ACS commission on Cancer are recognized for their excellence in cancer care. These designations indicate that the hospital meets rigorous standards for cancer treatment, research, and patient care, suggesting high-quality structural capabilities in oncology.
   (ii) Nurse Magnet Status: This status awarded by the American Nurse Credentialing Center (ANCC) is a prestigious recognition of a hospital's excellence in nursing practice and patient care. Achieving Magnet status indicates that a hospital has met rigorous standards for quality nursing, including superior staffing levels, advanced education, and training for nurses, and exemplary patient outcomes. Hospitals with Magnet status may receive higher scores in related performance categories positively influencing their overall ranking.
   (iii) NAEC-designated Epilepsy Center: This designation is provided by the National Association of Epilepsy Centers (NAEC) for providing comprehensive and specialized care for patients with epilepsy. These centers are evaluated based on their ability to deliver high-quality medical, surgical, and supportive care for individuals with epilepsy. Designation as an NAEC Epilepsy Center signifies that the hospital meets stringent criteria for specialized staff, advanced technology, multidisciplinary care, and outcome. Hospitals with this designation may receive higher scores in related performance categories positively influencing their overall ranking.
   (iv) NIA-Designated Alzheimer's Center: These centers are acknowledged for their research, advanced treatment options, and comprehensive care programs designed to address the complex needs of Alzheimer's patient and their families. Designation as an NIA-Designated Alzheimer's Center indicates that the hospital meets high standards in scientific research, clinical care, and community outreach. Hospitals with this designation may receive higher scores in related performance categories positively influencing their overall ranking.
   (v) FACT accreditation: This accreditation involves rigorous evaluation of clinical, laboratory, and collection facilities to ensure compliance with quality and safety standards. Hospitals with FACT accreditation are recognized for their excellence in cellular therapy, which may include maintaining a high level of patient care, adhering to stringent protocols, and demonstrating superior outcomes. Hospitals with this designation may receive higher scores in related performance categories positively influencing their overall ranking.
   (vi) CARF accreditation: Accreditation from the Commission on Accreditation of Rehabilitation Facilities (CARF International) designates a center as meeting standards of excellence in rehabilitation care. This accreditation involves a comprehensive evaluation of the facility's program, services, and adherence to best practices in patient care and rehabilitation outcomes. Hospitals with CAF accreditation may receive higher scores in related performance categories positively influencing their overall ranking.
   (vi) Rehabilitation Model Systems (RMS): Designation as a model system in rehabilitation by the National Institute on Disability, Independent Living, and Rehabilitation Research (NIDILRR). These systems are recognized for their excellence in research clinical care, and education in the field of rehabilitation. In the hospital ranking method, designation as an RMS center may be a critical performance criterion, reflecting the hospital's commitment to superior rehabilitation services and cutting-edge research. Hospitals with RMS designation may receive higher scores in related performance categories positively influencing their overall ranking.

In one instance, the scoring module 109 may synthesize the weighted data from various criteria into comprehensive performance scores for each hospital. The scoring module 109 may integrate individual scores from diverse factors such as hospital structure, nurse staffing levels, advanced technology availability, patient volumes, survey results, trauma center capabilities, or expert opinions. By applying the predefined weights to each criterion, the scoring module 109 may calculate specific scores, such as survival score, discharge to home score, and patient experience score, leveraging statistical methodologies to derive meaningful conclusions. In one example:
1. Survival score: The scoring module 109 may quantify a hospital's effectiveness in saving patient's lives. This score may be derived from data on patient outcomes, specifically focusing on survival rates for various medical conditions and procedures. The scoring module 109 may account for factors such as the severity of illnesses, the complexity of surgeries, and the effectiveness of emergency care. Hospitals with higher survival rates may be assigned higher scores, reflecting their proficiency in providing life-saving treatments and high-quality care.
2. Discharge from home score: The discharge to home measure assesses how well a hospital does at managing to discharge patients to home rather than sending them on to another acute or post-acute care setting following hospitalization. The scoring module 109 may measure a hospital's success in rehabilitating patients to the point where they can safely return to their homes rather than being transferred to another care facility. This score may be calculated based on the proportion of patients discharged directly to their homes compared to those discharged to rehabilitation centers, long-term care facilities, or other institutions. A higher discharge to home score may indicate that a hospital is effective in its treatment protocols, recovery programs, and post-discharge planning, ensuring patients achieve optimal recovery outcomes.
3. Patient experience score: The scoring module 109 may quantify the quality of care from the patient's perspective, incorporating feedback on various aspects of their hospital stay. This score may be derived from patient satisfaction surveys, which may assess factors such as communication with healthcare providers, the responsiveness of hospital staff, the cleanliness of facilities, and overall patient comfort and satisfaction. A higher patient experience score may indicate that a hospital excels in delivering patient-centered care and maintaining high standards of service.
4. Prevention of outpatient complications for Orthopedics and Urology: This measure evaluates the ability of hospitals to prevent complications related to procedures conducted on an outpatient basis. In some surgical specialties, outpatient procedures have long been routine. In others, surgeries that historically involved admitting patients to an inpatient setting are now increasingly performed on an outpatient basis. To reflect the growing role of outpatient procedural care, measures of outpatient procedural outcomes were introduced in Orthopedics and Urology. In one instance, the measure evaluates the ability of hospitals to successfully perform procedures without complications using an observed to expected ratio of potentially preventable complications. Each hospital's observed complication count is calculated as the total number of outpatient procedures with a subsequent clinically relevant complication within 30 days across all PSGs assigned to the specialty.
5. Readmission rate score: The scoring module 109 may assess the frequency with which patients need to be readmitted shortly after discharge, reflecting the hospital's effectiveness in providing long-term care.
6. Staff satisfaction score: The scoring module 109 may evaluate the morale and well-being of healthcare providers, influencing the quality of patient care.
7. Cost efficiency score: The scoring module 109 may assess the hospital's ability to deliver high-quality care while managing expenses, reflecting the economic sustainability of the hospital's operations.

The scoring module 109 may aggregate these individual scores into an overall score for each hospital, facilitating a clear and comparative assessment of hospital quality and effectiveness. This aggregation process may involve applying predefined weights to each criterion to ensure that the overall score accurately reflects the hospital's performance across multiple dimensions of care and operations. It should be understood that the scoring module 109 may calculate various other scores encompassing a wide range of performance indicators to provide a holistic evaluation of hospital quality.

In one instance, the ranking algorithm 111 may order hospitals based on the comprehensive scores generated by the scoring module 109. For example, once the scoring module 109 has calculated and aggregated the weighted scores for each criterion, the ranking algorithm 111 may take these overall scores and organize the hospitals into a ranked list. This list may reflect each hospital's performance across various dimensions such as clinical outcomes, patient satisfaction, advanced technology utilization, and specialized services. In one instance, the ranking algorithm 111 may systematically analyze data from various sources, including patient outcomes, healthcare quality metrics, and procedural success rates, to generate comprehensive rankings. In one example, the ranking algorithm 111 may analyze data such as patient mortality rates, complication rates, readmission rates, and patient satisfaction scores. Hospitals may earn scores based on their performance, with higher scores awarded for exceptional outcomes and penalization for below-average results. By integrating these scores, the ranking algorithm 111 may provide a comprehensive and objective ranking of hospitals, highlighting those that consistently deliver high-quality care across a broad spectrum of medical and surgical services.

In one instance, the machine-learning module 113 may leverage advanced algorithms and models to analyze complex healthcare data and generate accurate performance rankings. The machine-learning module 113 may employ a variety of machine-learning techniques including supervised learning algorithms like regression models, decision, trees, and ensemble methods (e.g., Random Forests, Gradient Boosting Machines) that utilizes training data, e.g., training data 412 illustrated in the training flow chart 400, for training a machine-learning model configured to rank a plurality of hospitals. The machine-learning module 113 may perform model training using training data, e.g., data from other modules, that contains input and correct output, to allow the model to learn over time. The training is performed based on the deviation of a processed result from a documented result when the inputs are fed into the machine-learning model, e.g., an algorithm measures its accuracy through the loss function, adjusting until the error has been sufficiently minimized. The machine-learning module 113 may also employ unsupervised learning techniques such as clustering and dimensionality reduction.

In one example, these algorithms are trained on historical data from diverse sources, allowing the system to identify patterns, predict outcomes, and adjust for risk factors. The machine-learning module 113 may incorporate feature engineering processes to extract and transform relevant data points, enhancing the predictive power of the models. Additionally, the machine-learning module 113 may utilize techniques like cross-validation and hyperparameter tuning to optimize model performance and ensure robustness. By continuously learning from new data inputs and feedback loops, the machine-learning module 113 may adapt to evolving trends in healthcare, ensuring the hospital rankings remain accurate, reliable, and reflective of current performance metrics. This dynamic capability enables the system to provide stakeholders with up-to-date and actionable insights for informed decision-making.

In one instance, the machine-learning module 113 may leverage deep learning to process unstructured data, including medical images and free-text clinical notes, using Convolutional Neural Networks (CNNs) and Recurrent Neural Networks (RNNs). NLP may extract valuable insights from patient reviews and physicians' notes, enriching the performance evaluation. The machine-learning module 113 may utilize transfer learning for fine-tuning pre-trained models on specific hospital data, enhancing performance with reduced computational resources. The machine-learning module 113 may implement temporal analysis to capture trends and changes over time, ensuring rankings remain current and relevant, while anomaly detection algorithms identify outliers and unusual patterns, maintaining the robustness of the system.

In one instance, the visualization module 115 may transform complex data and model outputs into intuitive and actionable insights. The visualization module 115 may utilize advanced data analytics techniques to perform in-depth analysis, uncovering patterns, trends, and correlations within the healthcare data. The visualization module 115 may generate interactive dashboards and displays to allow users to explore the data dynamically, facilitating a deeper understanding of hospital performance across various metrics. In one example, key performance indicators (KPIs) may be displayed through visually engaging charts, graphs, and heatmaps, enabling stakeholders to quickly identify strengths and areas for improvement. Additionally, the visualization module 115 may support drill-down capabilities, allowing users to delve into granular data for specific procedures, conditions, or demographic groups. In one example, real-time analytics features may provide up-to-date information, reflecting the latest data inputs and model updates. By making complex data accessible and understandable, the visualization module 115 may empower healthcare providers and administrators to make informed, data-driven decisions aimed at improving hospital quality and patient outcomes.

In one instance, the database 117 may store, manage, and retrieve vast amounts of healthcare data with high efficiency and reliability. The database 117 may employ advanced database technologies, such as relational databases (e.g., MySQL) for structured data and NoSQL databases for unstructured and semi-structured data, ensuring optimal performance and scalability. The database 117 may support complex queries and transactions, allowing for real-time data analytics and rapid access to critical information. The database 117 may implement robust indexing and partitioning strategies to enhance query performance and manage large datasets effectively. Additionally, the database 117 may integrate backup and disaster recovery solutions to ensure data integrity and availability in the event of system failure. Data integrity is further maintained through constraints, triggers, and stored procedures, enforcing business rules and data validation. By leveraging these advanced database management practices, the database 117 may provide a solid foundation for the analysis platform 101, enabling it to handle the complexity and scale of healthcare data with ease.

In one instance, various elements of the system 100 may communicate with each other through the communication network 119. The communication network 119 may support a variety of different communication protocols and communication techniques. In one embodiment, the communication network 119 may allow data source(s) 121 to communicate with the analysis platform 101. The communication network 119 of the system 100 may include one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

In one instance, data source(s) 121 may encompass a wide array of datasets essential for comprehensive performance assessment of the hospitals. These sources may include publicly available indicators, such as Medicare's Hospital Compare data, which may provide insight into hospital quality measures like mortality rates, readmission rates, and patient safety indicators. The MBSF and LDS SAF may offer detailed patient-level information crucial for risk adjustment and outcome analysis. Additionally, the American Hospital Association (AHA) surveys may contribute data on hospital characteristics and operational metrics, while the Hospital Consumer Assessment of Healthcare Providers and Systems (HCAHPS) survey may capture patient-reported experiences and satisfaction. In one instance, specialized datasets like those from the American Board of Orthopedic Surgery verification data may provide specific performance metrics related to orthopedic care quality. Integrating these diverse data sources may involve rigorous data governance and integration processes to ensure data accuracy, consistency, and interoperability across different formats and sources. By leveraging these comprehensive data sources, the analysis platform 101 may provide a nuanced evaluation of the hospital's performance across various dimensions, thereby facilitating information decision-making.

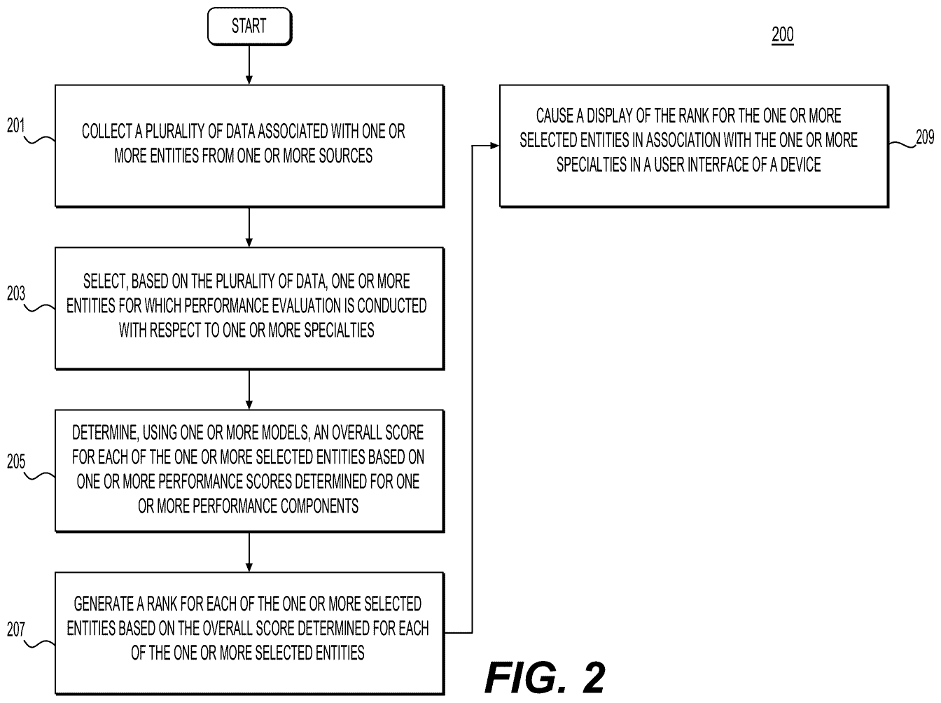

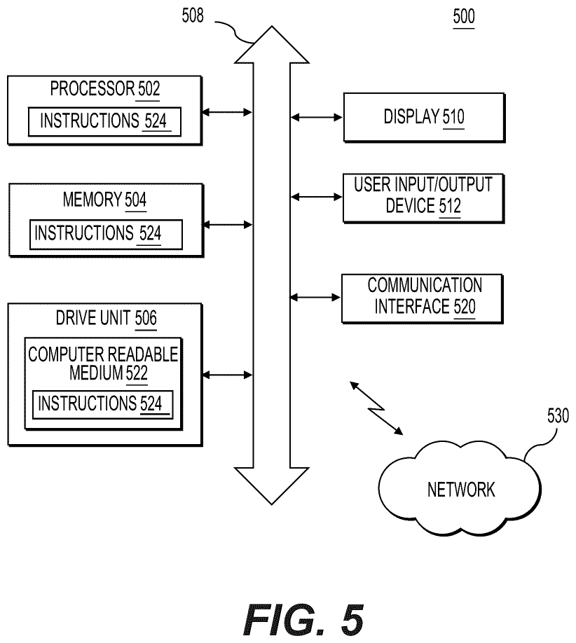

FIG. 2 is a flowchart of a process for ranking entities based on performance evaluation with respect to specialties, according to aspects of the disclosure. In various embodiments, the analysis platform 101 and/or any of the modules 103-115 may perform one or more portions of the process 200 and are implemented using, for instance, a chip set including a processor and a memory as shown in FIG. 5. As such, the analysis platform 101 and/or any of modules 103-115 may provide means for accomplishing various parts of the process 200, as well as means for accomplishing embodiments of other processes described herein in conjunction with other components of the system 100. Although the process 200 is illustrated and described as a sequence of steps, it is contemplated that various embodiments of the process 200 may be performed in any order or combination and need not include all of the illustrated steps.

In step 201, the analysis platform 101 may collect a plurality of data associated with one or more entities (e.g., hospitals) from one or more sources. In one example, one or more sources may include government healthcare databases, hospital internal records, external accreditation entities, healthcare providers surveys, patient feedback, publicly available healthcare performance reports, or research publications. The integration of these varied data points may ensure a holistic view of hospital performance, allowing for more accurate and reliable quality assessment by capturing a wide range of metrics and perspectives relevant to healthcare delivery.

In step 203, the analysis platform 101 may select, based on the plurality of data, one or more entities for which performance evaluation is conducted with respect to one or more specialties. The one or more specialties may include one or more of: cancer; cardiology, heart and vascular surgery; diabetes and endocrinology; ear, nose, and throat; gastroenterology and GI surgery; geriatrics, obstetrics and gynecology; neurology and neurosurgery; ophthalmology; pulmonology and lung surgery; psychiatry; rehabilitation; rheumatology; or urology. One or more entities may be selected based on one or more of: structural characteristics, volume, or discharge characteristics. In one example, the structural characteristics may refer to physical and organizational attributes of the hospitals, such as the number of beds, presence of advanced medical technologies, availability of specialized units, staffing level, and so on. In one example, the volume may pertain to the hospital's capacity and experience, measured by the volume of patients treated in specific departments or for particular conditions. Higher patient volumes may indicate a greater level of expertise and efficiency in handling specific medical cases. In one example, discharge characteristics may indicate data relating to patient discharges, including discharge rates, average length of stay, discharge destination (e.g., home, rehabilitation centers, or other medical facilities), and readmission rates. In one instance, if the discharge characteristic for an entity is below a pre-determined threshold, the entity may be selected if nominated by a certain percentage or number of providers and/or provider systems.

In step 205, for each of the one or more specialties, the analysis platform 101 may determine, using one or more models, an overall score for each of the one or more selected entities based on one or more performance scores determined for one or more performance components. The one or more performance components may include one or more of a structural component, a process/expert opinion component, an outcome component, a patient experience component, or a public transparency component, wherein each component includes one or more performance indicators. One or more models may include a scoring model. In one example, the scoring model may assess hospital performance on one or more performance components and may assign weights and/or scores to each of the components.

In one example, the performance score for the structural component may be determined based on the number and type of medical facilities, availability of advanced medical technologies, staffing level and qualifications, and so on. In one example, the performance score for the process/expert opinion component may be determined based on clinical practices, administrative procedures, adherence to best practice guidelines, the quality of patient care processes, or expert evaluations. These expert opinions may be gathered from experienced healthcare providers, who may provide qualitative assessments based on their knowledge and experience. In one example, the performance score for the outcome component may be determined based on survival rates, discharge to home rates, patient satisfaction scores, readmission rates, or complications rates. In one example, the performance score for the patient experience component may be determined based on a plurality of data objects from one or more of patients, entity leaders, or other stakeholders during a pre-determined time period. Such plurality of data objects from the patients, entity leaders, or other stakeholders may include survey results. In one example, the performance score for the public transparency component may be determined based on data voluntarily reported to the public by the corresponding entity or participation by the entities in clinical registries and other public transparency programs that may require the hospitals to share detailed information about their clinical practices, outcomes, and quality measures. Hospitals that actively engage in these transparency efforts are demonstrating a commitment to accountability and quality improvement.

With respect to the specialties of cancer, diabetes and endocrinology, ear, nose, and throat, gastroenterology and GI surgery, geriatrics, ophthalmology, psychiatry, rheumatology, and urology, the one or more scoring models may use the performance scores for the structural component, process/expert opinion component, outcome component, and patient experience component. For the specialties of cardiology, heart and vascular surgery, obstetrics and gynecology, neurology and neurosurgery, and pulmonology and lung surgery, the one or more scoring models may use the performance scores for the structural component, process/expert opinion component, outcome component, patient experience component, and public transparency component. For the specialty of rehabilitation, the one or more scoring models may use the performance scores for the structural component, process component, and outcome component.

In one instance, each of the one or more performance indicators associated with a corresponding performance component represents an attribute or a trait of a corresponding entity that is used in evaluating performance of that entity. The one or more performance indicators for the structural component may include one or more of: advanced technologies; number of patients; outpatient volume; volume of care; nurse staffing; trauma center; patient services; ICU specialists; designated institution; nurse magnet status; or accreditation. The one or more performance indicators for the outcome component may include one or more of: mortality rate; discharge rate; or measure of outpatient complication.

In one embodiment, the analysis platform 101 determines at least the performance scores for the structural component and the outcome component in the following manner, for each of these performance scores. The analysis platform 101 first determines one or more values representative of corresponding one or more performance indicators. In one example, the analysis platform 101 may identify specific metric and indicators that may reflect the quality and capabilities of hospital structures (e.g., advanced technologies; number of patients; outpatient volume; volume of care; nurse staffing; trauma center; patient services; ICU specialists; designated institution; nurse magnet status; or accreditation, etc.) and outcomes (e.g., mortality rates, discharge rate, complications rate, etc.) relevant to each component. Once the one or more values are determined. The analysis platform 101 may then normalize the one or more values representative of corresponding one or more performance indicators. The analysis platform 101 may then assign a weight to each of the one or more performance indicators. In one example, the weights may be assigned based on their relative importance in assessing the structural or outcome components. These weights may reflect the impact of each indicator on the overall performance score. The analysis platform 101 may then generate a normalized score for each of one or more performance indicators based on the corresponding weight and normalized value. In one example, the normalized score for each performance indicator may be calculated by multiplying the normalized value by its assigned weight. This step integrates the normalized data with the weighted importance of each indicator. The analysis platform 101 may then generate a performance score for the corresponding performance component based on the normalized score(s) for one or more performance indicators. In one example, the normalized score of all performance indicators may be aggregated within the structural or outcome component to generate an overall performance score. This score may represent the hospital's performance level in terms of structural capabilities or outcomes, providing a comprehensive assessment for stakeholders in healthcare decision-making and quality improvement efforts.

In one embodiment, the analysis platform 101 may determine the overall score for each of the one or more selected entities in the following manner. The analysis platform 101 assigns a weight to each of the one or more performance components, wherein the performance score for each of the one or more performance components may be based on the corresponding weight. In one example, the analysis platform 101 may assign weight to each performance component (e.g., structural, process/expert opinion, outcomes, patient experiences) based on their relative importance in evaluating overall hospital performance. Upon assigning the weight, the analysis platform 101 may aggregate the one or more performance scores for the one or more performance components, to determine the overall score. In one example, the performance scores from all selected components may be aggregated by applying the assigned weights. This aggregation may combine the weighted scores of structural, process/expert opinion, outcomes, and patient experiences components into a single composite score for each entity. The result is an overall score that provides a comprehensive assessment of the entity's performance relative to others within the evaluation framework.

In one instance, the analysis platform 101 may determine the performance score for the process/expert opinion component in the following manner. The analysis platform 101 may receive a plurality of data objects from qualified providers and/or provider systems during a pre-determined time period. The plurality of data objects received from the qualified providers and/or provider systems include survey responses. In one example, the analysis platform 101 may collect a diverse set of data objects (e.g., survey responses, reviews, expert opinions, or other qualitative inputs) from qualified healthcare providers or provider systems over a pre-determined period. The analysis platform 101 may then determine a score for each data object received from a corresponding provider or provider system. In one example, the analysis platform may assess each data object received from providers or systems to assign a score based on pre-determined evaluation criteria. This step may involve evaluating the quality, relevance, and impact of the information provided. The analysis platform 101 may then generate a weighted score for each data object based on one or more characteristics of the corresponding provider or provider system. In one example, the analysis platform 101 may apply weights to each data object based on the characteristics and credibility of the corresponding provider or provider system. Providers with recognized expertise or specialized qualifications may receive higher weights, reflecting their influence on the overall performance score. The analysis platform 101 may then generate transformed scores by applying log transformation to the weighted scores for the plurality of data objects. In one example, the logarithmic transformation may be used to normalize the distribution of scores and address potential outliers, ensuring a more balanced representation of performance across data objects. The analysis platform 101 may then generate normalized scores by normalizing the transformed scores. In one example, the transformed scores may be normalized to establish a consistent scale and comparability among different data objects. Normalization may adjust for variations in scoring ranges and may standardize the scores across all evaluated providers or systems. Then, the analysis platform 101 may generate a performance score for the process/expert opinion component based on the normalized scores for the data objects. In one example, normalized scores for all data objects within the process/expert opinion component may be aggregated to calculate an overall performance score. This score may reflect the collective assessment of healthcare processes, clinical expertise, and expert opinions contributed by qualified providers or provider systems. It may provide a comprehensive evaluation of the hospital's procedural quality and expert guidance, supporting informed decision-making.

With continuing reference to FIG. 2, in step 207, the analysis platform 101 may generate a rank for each of one or more selected entities based on the overall score determined for each of the one or more selected entities. In one example, the overall scores may be calculated for each selected entity by aggregating the performance scores across all relevant performance components. Once the overall scores are calculated for all selected entities, the entities are ranked based on their respective scores. Entities with higher overall scores may typically receive higher ranks, indicating superior performance compared to those with lower scores. A rank list may be generated where entities are sequentially arranged from highest to lowest score.

In step 209, the analysis platform 101 may cause a display of the rank for one or more selected entities in association with one or more specialties in the user interface of a device. In one example, the rank may be presented in a clear and accessible format, such as a table or dashboard, to facilitate easy interpretation and comparison.

FIG. 3 is a flowchart of a process for determining eligibility and ranking of community hospitals in a data-driven ranking system, according to aspects of the disclosure. In various embodiments, the analysis platform 101 and/or any of the modules 103-115 may perform one or more portions of the process 300 and are implemented using, for instance, a chip set including a processor and a memory as shown in FIG. 5. As such, the analysis platform 101 and/or any of modules 103-115 may provide means for accomplishing various parts of the process 300, as well as means for accomplishing embodiments of other processes described herein in conjunction with other components of the system 100. Although the process 300 is illustrated and described as a sequence of steps, it is contemplated that various embodiments of the process 300 may be performed in any order or combination and need not include all of the illustrated steps.

In step 301, the analysis platform 101 may determine the eligibility of community hospitals from the fiscal year 2021. The data sourced from the American Hospital Association (AHA) database may include 4,515 hospitals. Eligibility is assessed based on four specific criteria:
1. Membership in the counsel of Teaching Hospitals (COTH);
2. Affiliation with a medical school recognized by the American Medical Association (AMA) or the American Osteopathic Association (AOA); or
3. Having at least 200 hospital beds; or
4. Having at least 100 beds along with at least four out of eight key advanced technologies.

Hospitals meeting any of these criteria may be deemed eligible for ranking. As a result, 2,320 hospitals qualify for further evaluation, while 2,195 hospitals do not meet the eligibility criteria and are eliminated from analysis (step 319).

In step 303, the analysis platform 101 may check whether the qualified hospitals responded to the AHA annual survey for fiscal year 2021. This may ensure that the most recent and relevant data is used for evaluation. If the hospitals responded to the FY2021 AHA annual survey, proceed to step 305. If the hospitals did not respond to the FY2021 AHA annual survey, proceed to step 307.

In step 305, the analysis platform 101 may compile data including nomination and information from other ranking databases into the final universe file for the hospitals that responded to the FY2021 AHA annual survey. This comprehensive dataset may include all inputs for the detailed analysis and scoring that follow in the ranking process.

In step 307, the analysis platform 101 may check whether the hospitals that did not respond to the FY2021 AHA annual survey responded to the AHA survey in fiscal years 2020 and 2019. This may ensure that hospitals with consistent historical data sets are still considered in the ranking process despite missing the most recent survey. The hospitals that responded to the AHA survey in fiscal years 2020 and 2019 may proceed to step 309. The hospitals that did not respond to the AHA survey in fiscal years 2020 and 2019 may proceed to step 311.

In step 309, the analysis platform 101 may determine that the hospitals that did not respond to the FY2021 AHA annual survey, did respond in the fiscal year 2020. The hospitals are then included in the final universe file for ranking purposes. This file consolidates nominations, FY2020 AHA data, and other relevant rankings data, ensuring comprehensive and consistent inputs for the subsequent analysis and scoring stages of the hospital ranking process.

In step 311, the analysis platform 101 may include the hospitals that did not respond to the AHA surveys for FY2021, FY2020, or FY 2019 in the final universe file with only nominations and other rankings data. While these hospitals may lack recent AHA data, they are still considered for ranking based on other available information and nominations received.

In step 313, the analysis platform 101 may assess whether each hospital has a sufficient number of discharges per specialty to ensure that hospitals have adequate patient volume in various specialties to provide reliable and representative data for ranking. If the hospitals meet the threshold for sufficient discharges per specialty (e.g., N=1,899), it proceeds to step 317. However, hospitals that do not meet the sufficient discharge threshold (e.g., N=421) proceed to step 315 for further evaluation.

In step 315, the analysis platform 101 may evaluate the hospitals that do not meet the sufficient discharge threshold based on their expert opinion scores. If these hospitals have an expert opinion score of 1% or greater (e.g., N=7), they are reconsidered for eligibility and proceed to step 319. On the other hand, if these hospitals do not have an expert opinion score of 1% or greater (e.g., N=412) they are eliminated from analysis (step 319).

In step 317, the analysis platform 101 may consider the hospitals that meet the threshold for sufficient discharges per specialty (e.g., N=1,899) and the hospitals with an expert opinion score of 1% or greater (e.g., N=7). The analysis platform 101 may evaluate these hospitals in the 11 HQ-driven specialties (excluding rehabilitation), and may generate rankings for these total eligible hospitals (e.g., N=1,906).

In such a manner, the analysis platform 101 may determine the eligibility of hospitals for inclusion in a comprehensive ranking system. This approach may ensure that only hospitals with sufficient and reliable data, and recognized expertise, are ranked thereby providing a transparent, accurate, and meaningful comparison of hospital performance across various quality indicators.

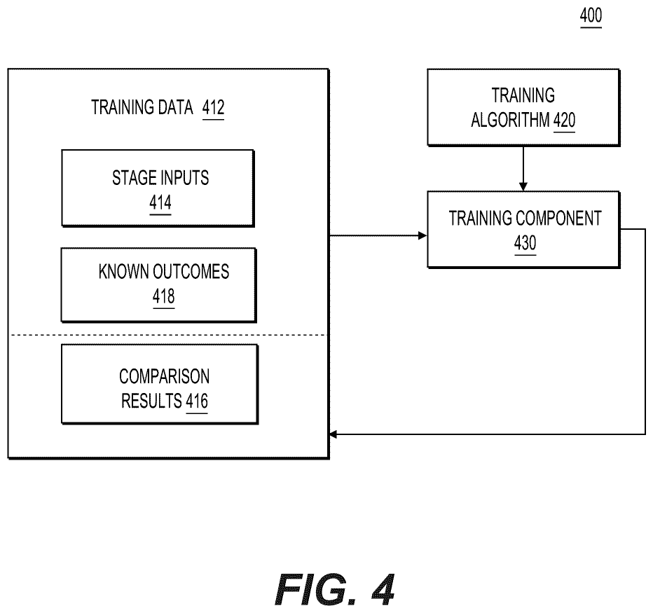

One or more implementations disclosed herein include and/or may be implemented using a machine-learning model. For example, one or more of the modules of the analysis platform 101 may be implemented using a machine-learning model and/or may be used to train the machine-learning model. A given machine-learning model may be trained using the training flow chart 400 of FIG. 4. Training data 412 may include one or more of stage inputs 414 and known outcomes 418 related to the machine-learning model to be trained. The stage inputs 414 may be from any applicable source including text, visual representations, data, values, comparisons, stage outputs, e.g., one or more outputs from one or more actions or operations from FIG. 2. The known outcomes 418 may be included for the machine-learning models generated based upon supervised or semi-supervised training. An unsupervised machine-learning model may not be trained using known outcomes 418. Known outcomes 418 may include known or desired outputs for future inputs similar to, or in the same category as, stage inputs 414 that do not have corresponding known outputs.

The training data 412 and a training algorithm 420, e.g., one or more of the modules implemented using the machine-learning model and/or may be used to train the machine-learning model, may be provided to a training component 430 that may apply the training data 412 to the training algorithm 420 to generate the machine-learning model. According to an implementation, the training component 430 may be provided comparison results 416 that compare a previous output of the corresponding machine-learning model to apply the previous result to re-train the machine-learning model. The comparison results 416 may be used by training component 430 to update the corresponding machine-learning model. The training algorithm 420 may utilize machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, models specifically discussed in the present disclosure, or the like.

The machine-learning model used herein may be trained and/or used by adjusting one or more weights and/or one or more layers of the machine-learning model. For example, during training, a given weight may be adjusted (e.g., increased, decreased, removed) based upon training data or input data. Similarly, a layer may be updated, added, or removed based upon training data/and or input data. The resulting outputs may be adjusted based upon the adjusted weights and/or layers.

In general, any process or operation discussed in this disclosure is understood to be computer-implementable, such as the processes illustrated in FIG. 2 may be performed by one or more processors of a computer system as described herein. A process or process step performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

FIG. 5 illustrates an implementation of a computer system that may execute techniques presented herein. The computer system 500 can include a set of instructions that can be executed to cause the computer system 500 to perform any one or more of the methods or computer based functions disclosed herein. The computer system 500 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In a networked deployment, the computer system 500 may operate in the capacity of a server or as a client user computer in a server-client user network environment, or as a peer computer system in a peer-to-peer (or distributed) network environment. The computer system 500 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 can be implemented using electronic devices that provide voice, video, or data communication. Further, while the computer system 500 is illustrated as a single system, the term “system” shall also be taken to include any collection of systems or sub-systems that individually or jointly execute a set, or multiple sets, of instructions to perform one or more computer functions.

As illustrated in FIG. 5, the computer system 500 may include a processor 502, e.g., a central processing unit (CPU), a graphics processing unit (GPU), or both. The processor 502 may be a component in a variety of systems. For example, the processor 502 may be part of a standard personal computer or a workstation. The processor 502 may be one or more processors, digital signal processors, application specific integrated circuits, field programmable gate arrays, servers, networks, digital circuits, analog circuits, combinations thereof, or other now known or later developed devices for analyzing and processing data. The processor 502 may implement a software program, such as code generated manually (i.e., programmed).

The computer system 500 may include a memory 504 that can communicate via bus 508. The memory 504 may be a main memory, a static memory, or a dynamic memory. The memory 504 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts or tasks illustrated in the figures or described herein may be performed by the processor 502 executing the instructions stored in the memory 504. The functions, acts, or tasks are independent of the particular type of instruction set, storage media, processor or processing strategy and may be performed by software, hardware, integrated circuits, firmware, micro-code and the like, operating alone or in combination. Likewise, processing strategies may include multiprocessing, multitasking, parallel processing, and the like.

As shown, the computer system 500 may further include a display 510, such as a liquid crystal display (LCD), an organic light emitting diode (OLED), a flat panel display, a solid-state display, a cathode ray tube (CRT), a projector, a printer or other now known or later developed display device for outputting determined information. The display 510 may act as an interface for the user to see the functioning of the processor 502, or specifically as an interface with the software stored in the memory 504 or in the drive unit 506.

Additionally or alternatively, the computer system 500 may include an input/output device 512 configured to allow a user to interact with any of the components of the computer system 500. The input/output device 512 may be a number pad, a keyboard, or a cursor control device, such as a mouse, or a joystick, touch screen display, remote control, or any other device operative to interact with the computer system 500.

The computer system 500 may also or alternatively include drive unit 506 implemented as a disk or optical drive. The drive unit 506 may include a computer-readable medium 522 in which one or more sets of instructions 524, e.g. software, can be embedded. Further, instructions 524 may embody one or more of the methods or logic as described herein. The instructions 524 may reside completely or partially within the memory 504 and/or within the processor 502 during execution by the computer system 500. The memory 504 and the processor 502 also may include computer-readable media as discussed above.

In some systems, computer-readable medium 522 includes the set of instructions 524 or receives and executes the set of instructions 524 responsive to a propagated signal so that a device connected to network 530 can communicate voice, video, audio, images, or any other data over the network 530. Further, the set of instructions 524 may be transmitted or received over the network 530 via communication port or interface 520, and/or using bus 508. The communication port or interface 520 may be a part of the processor 502 or may be a separate component. The communication port or interface 520 may be created in software or may be a physical connection in hardware. The communication port or interface 520 may be configured to connect with a network 530, external media, the display 510, or any other components in computer system 500, or combinations thereof. The connection with the network 530 may be a physical connection, such as a wired Ethernet connection or may be established wirelessly as discussed below. Likewise, the additional connections with other components of the computer system 500 may be physical connections or may be established wirelessly. The network 530 may alternatively be directly connected to the bus 508.

While the computer-readable medium 522 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that causes a computer system to perform any one or more of the methods or operations disclosed herein. The computer-readable medium 522 may be non-transitory, and may be tangible.

The computer-readable medium 522 can include a solid-state memory such as a memory card or other package that houses one or more non-volatile read-only memories. The computer-readable medium 522 can be a random-access memory or other volatile re-writable memory. Additionally or alternatively, the computer-readable medium 522 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

Computer system 500 may be connected to network 530. The network 530 may define one or more networks including wired or wireless networks. The wireless network may be a cellular telephone network, an 802.10, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 530 may include wide area networks (WAN), such as the Internet, local area networks (LAN), campus area networks, metropolitan area networks, a direct connection such as through a Universal Serial Bus (USB) port, or any other networks that may allow for data communication. The network 530 may be configured to couple one computing device to another computing device to enable communication of data between the devices. The network 530 may generally be enabled to employ any form of machine-readable media for communicating information from one device to another. The network 530 may include communication methods by which information may travel between computing devices. The network 530 may be divided into sub-networks. The sub-networks may allow access to all of the other components connected thereto or the sub-networks may restrict access between the components. The network 530 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of the present disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

What is claimed is:

1. A computer-implemented method comprising:
collecting, using one or more processors, a plurality of data associated with one or more entities from one or more sources;
selecting, using the one or more processors and based on the plurality of data, one or more entities for which performance evaluation is conducted with respect to one or more specialties;
for each of the one or more specialties:
determining, using the one or more processors and using one or more models, an overall score for each of the one or more selected entities based on one or more performance scores determined for one or more performance components, wherein the one or more performance components include one or more of:
a structural component,
a process/expert opinion component,
an outcome component,
a patient experience component, or
a public transparency component, wherein each component includes one or more performance indicators; and
generating, using the one or more processors, a rank for each of the one or more selected entities based on the overall score determined for each of the one or more selected entities; and
causing, using the one or more processors, a display of the rank for the one or more selected entities in association with the one or more specialties in a user interface of a device.

2. The computer-implemented method of claim 1, the one or more entities are selected based on one or more of structural characteristics, volume, or discharge characteristics.

3. The computer-implemented method of claim 2, wherein, if the discharge characteristics for an entity is below a pre-determined threshold, the entity is selected if the entity is nominated by a certain percentage or number of providers and/or provider systems.

4. The computer-implemented method of claim 1, wherein at least the performance scores for the structural component and the outcome component are determined by, for each of these performance scores:
determining one or more values representative of corresponding one or more performance indicators;
normalizing the one or more values;
assigning a weight to each of the one or more performance indicators;
generating a normalized score for each of the one or more performance indicators based on the corresponding weight and normalized value; and
generating a performance score for the corresponding performance component based on the normalized score for the one or more performance indicators.

5. The computer-implemented method of claim 1, wherein determining the overall score for each of the one or more selected entities comprises:
assigning a weight to each of the one or more performance components, wherein the performance score for each of the one or more performance components is based on the corresponding weight; and
aggregating the one or more performance scores for the one or more performance components.

6. The computer-implemented method of claim 1, wherein at least the performance score for the process/expert opinion component is determined by:
receiving a plurality of data objects from qualified providers and/or provider systems during a pre-determined time period;
determining a score for each data object received from a corresponding provider or provider system;
generating a weighted score for each data object based on one or more characteristics of the corresponding provider or provider system;
generating transformed scores by applying log transformation to the weighted scores for the plurality of data objects;
generating normalized scores by normalizing the transformed scores; and
generating a performance score for the process/expert opinion component based on the normalized scores for the data objects.

7. The computer-implemented method of claim 6, wherein the plurality of data objects received from the qualified providers and/or provider systems include survey responses.

8. The computer-implemented method of claim 1, wherein the performance score for the patient experience component is determined based on a plurality of data objects from one or more of patients, entity leaders, or other stakeholders during a pre-determined time period.

9. The computer-implemented method of claim 8, wherein the plurality of data objects received from one or more of the patients, entity leaders, or other stakeholders include survey results.

10. The computer-implemented method of claim 1, wherein the performance score for the public transparency component is determined based on data voluntarily reported to the public by a corresponding entity.

11. The computer-implemented method of claim 1, wherein the one or more specialties include one or more of:
cancer;
cardiology, heart and vascular surgery;
diabetes and endocrinology;
ear, nose, and throat;
gastroenterology and GI surgery;
geriatrics,
obstetrics and gynecology;
neurology and neurosurgery;
ophthalmology;
pulmonology and lung surgery;
psychiatry;
rehabilitation;
rheumatology; or
urology.

12. The computer-implemented method of claim 11, wherein the one or more models include one or more scoring models.

13. The computer-implemented method of claim 12, wherein, with respect to the specialties of cancer, diabetes and endocrinology, ear, nose, and throat, gastroenterology and GI surgery, geriatrics, ophthalmology, psychiatry, rheumatology, and urology, the one or more scoring models use the performance scores for the structural component, process/expert opinion component, outcome component, and patient experience component.

14. The computer-implemented method of claim 12, wherein, for the specialties of cardiology, heart and vascular surgery, obstetrics and gynecology, neurology and neurosurgery, and pulmonology and lung surgery, the one or more scoring models use the performance scores for the structural component, process/expert opinion component, outcome component, patient experience component, and public transparency component.

15. The computer-implemented method of claim 12, wherein, for the specialty of rehabilitation, the one or more scoring models use the performance scores for the structural component, process component, and outcome component.

16. The computer-implemented method of claim 1, wherein each of the one or more performance indicators associated with a corresponding performance component represents an attribute or a trait of a corresponding entity that is used in evaluating performance of that entity.

17. The computer-implemented method of claim 1, wherein the one or more performance indicators for the structural component include one or more of:
advanced technologies;
number of patients;
outpatient volume;
volume of care;
nurse staffing;
trauma center;
patient services;
ICU specialists;
designated institution;
nurse magnet status; or
accreditation.

18. The computer-implemented method of claim 1, wherein the one or more performance indicators for the outcome component include one or more of:
mortality rate;
discharge rate; or
measure of outpatient complication.

19. A system comprising:
one or more processors of a computing system; and
at least one non-transitory computer readable medium storing instructions which, when executed by the one or more processors, cause the one or more processors to perform operations comprising:
collecting a plurality of data associated with one or more entities from one or more sources;
selecting, based on the plurality of data, one or more entities for which performance evaluation is conducted with respect to one or more specialties;
for each of the one or more specialties:
determining, using one or more models, an overall score for each of the one or more selected entities based on one or more performance scores determined for one or more performance components, wherein the one or more performance components include one or more of:
a structural component,
a process/expert opinion component,
an outcome component,
a patient experience component, or
a public transparency component, wherein each component includes one or more performance indicators; and
generating a rank for each of the one or more selected entities based on the overall score determined for each of the one or more selected entities; and
causing a display of the rank for the one or more selected entities in association with the one or more specialties in a user interface of a device.

20. A non-transitory computer readable medium, the non-transitory computer readable medium storing instructions which, when executed by one or more processors of a computing system, cause the one or more processors to perform operations comprising:
collecting a plurality of data associated with one or more entities from one or more sources;
selecting, based on the plurality of data, one or more entities for which performance evaluation is conducted with respect to one or more specialties;
for each of the one or more specialties:
determining, using one or more models, an overall score for each of the one or more selected entities based on one or more performance scores determined for one or more performance components, wherein the one or more performance components include one or more of:
a structural component,
a process/expert opinion component,
an outcome component,
a patient experience component, or
a public transparency component, wherein each component includes one or more performance indicators; and
generating a rank for each of the one or more selected entities based on the overall score determined for each of the one or more selected entities; and
causing a display of the rank for the one or more selected entities in association with the one or more specialties in a user interface of a device.
